(19) United States
(12) Patent Application Publication
(10) Pub. No.: US 2016/0334333 A1
(43) Pub. Date: Nov. 17, 2016

(54) SENSING FIBER AND SENSING DEVICE

(71) Applicant: I-SHOU UNIVERSITY, KAOHSIUNG CITY (TW)

(72) Inventors: Jung-Sheng Chiang, Kaohsiung City (TW); Nai-Hsiang Sun, Kaohsiung City (TW); Wen-Fung Liu, Taipei City (TW); Shih-Chiang Lin, Kaohsiung City (TW)

(21) Appl. No.: 14/842,843

(22) Filed: Sep. 2, 2015

(30) Foreign Application Priority Data
May 15, 2015 (TW) 104115562

(51) Int. Cl. G01N 21/552; G01N 33/543; G02B 6/02; G01N 21/65; G01N 21/64

(52) U.S. Cl. CPC G01N 21/554; G01N 21/658; G01N 21/648; G02B 6/02314; G01N 33/54373; G01N 2201/088; G01N 2201/068; G01N 2201/061

(57) ABSTRACT

A sensing fiber adapted to transmit a sensing light along a path and sense an object is provided. The fiber includes a core, a plurality of photonic crystal structures surrounding the core, a sensing surface and a metal sensing layer. The core is located at the center of the fiber. The photonic crystal structures extend along the path. The sensing surface extends along part of the path and adjacent to the core, and the metal sensing layer having a plurality of metal grating structure is disposed on the sensing surface. When the fiber is sensing the object, the metal sensing layer is located between the sensing surface and the object, and part of the sensing light will be converted into a signal light by the object on the metal sensor layer. A sensing device is also provided.

### CROSS REFERENCE TO RELATED APPLICATION

This application claims the priority benefit of Taiwan application serial no. 104115562, filed on May 15, 2015. The entirety of the above-mentioned patent application is hereby incorporated by reference herein and made a part of specification.

### BACKGROUND OF THE INVENTION

1. Field of the Invention

The invention relates to a fiber and an optical device, specifically to a sensing fiber and a sensing device.

2. Description of Related Art

Along with the development of science and technology, infoimation transfer has become one of the most promising science and technology to be developed. The capacity, stability, quality, and speed of infoll iation transmission always are the main topics of infoi nation transfer science and technology, so that the important role and the future development of the optical fiber communication are further highlighted and emphasized. In 1987, the two scientists Sajeev John and Eli Yablonovitch separately proposed the fundamental theory of photonic crystal structure having periodic property, and thus the refractive index or the dielectric constant of material changes periodically because of one dimensional, two dimensional, and three dimensional arrangement method. Furthermore, in 1996, Dr. Russell, Dr. J. C. Knight, et al., apply photonic crystal structure to the fiber by fabricating the cladding around the core of the fiber, and the cladding has a plurality of air holes periodically arranged, so as to form the photonic crystal fiber.

On the other hand, along with the development of science and technology, it was found in 1902 that the surface plasmon has been applied to many fields, such as chemical sensor, biomedical science, food examination, et al. The surface plasmon resonance is the coherence surface electromagnetic wave formed by free electrons existing on a metal and dielectric surface, the group behaviours of the free electrons is called as surface plasmon. When the surface plasmon is generated, the surface plasmon mode is limited to nearby the metal surface, and field strength of the electromagnetic wave on the metal surface and the dielectric surface has a maximum value, moves away from the metal surface, and presents a decreasing exponential property. This phenomenon shows a high sensitivity property of the surface plasmon, and thus be applied to measuring many types of surface spectroscopy, such as Surface-Enhanced Raman Spectroscopy (SERS).

However, application and sensitivity of the surface plasmon generated by the evanescent wave of the metal film is limited, and in the process of forming the metal film in the photonic crystal structure fiber having air holes, the shape of the air holes will be changed under high temperature condition, and it is very difficult to control the quality of the thickness of the metal film.

### SUMMARY OF THE INVENTION

The invention provides a sensing fiber which has a high sensitivity.

The invention provides a sensing device which can provide a good sensing effect.

A sensing fiber in one embodiment of the invention is adapted to transmit a sensing light along a path and senses an object. The sensing fiber includes a core, a plurality of photonic crystal structures surrounding the core, a sensing surface and a metal sensing layer. The core is located at the center of the sensing fiber. The photonic crystal structures extend along the path. The sensing surface extends along a part of the path and be adjacent to the core, and the metal sensing layer having a plurality of metal grating structures is disposed on the sensing surface. When the sensing fiber senses the object, the metal sensing layer is located between the sensing surface and the object, and a part of the sensing light is converted into a signal light by the object on the metal sensor layer.

In one embodiment of the invention, a sensing device is adapted to sense an object, the sensing device includes a light source, the aforementioned sensing fiber, and a receiving unit. The sensing fiber is adapted to transmit the sensing light along the path and senses the object. The sensing fiber further includes a light-entering end and a light-exiting end, and the sensing surface is located between the light-entering end and the light-exiting end. The sensing light emitted by the light source enters the sensing fiber from the light-entering end, a part of the sensing light is converted into a signal light by the object on the metal sensor layer, and the signal light is emitted from the light-exiting end and enters the receiving unit.

In one embodiment of the invention, the metal grating structures of the metal sensing layer are arranged along a direction perpendicular to the path.

In one embodiment of the invention, the metal sensing layer has a total thickness in a direction perpendicular to the sensing surface, and the total thickness is greater than or equal to 40 nm and less than or equal to 80 nm.

In one embodiment of the invention, the metal sensing layer further has a first metal layer and a second metal layer located between the sensing surface and the first metal layer. The metal grating structures are formed at the first metal layer.

In one embodiment of the invention, the metal grating structures conform with

$$0.02 \leq \frac{d}{Λ} \leq 0.04 ,$$

wherein d is a depth of the metal grating structures along a direction perpendicular to the sensing surface, A is a pitch of the metal grating structures.

In one embodiment of the invention, the receiving unit is an optical spectrum analyzer (OSA), a power meter, or a light meter.

Based on the above, the metal sensing layer on the sensing surface of the sensing fiber of the embodiments of the invention has the plurality of the metal grating structures. Therefore, when the sensing light is transmitted in the core, the sensing light can be effectively transmitted to the object on the metal sensing layer, and the signal light converted by the object is obtained to provide a good sensing effect. Because the sensing device of the embodiments of the invention has the sensing fiber, when the light source emits the sensing light to the sensing fiber, the receiving unit can all receive a good signal light of the object.

In order to make the aforementioned and other features and advantages of the invention comprehensible, several exemplary embodiments accompanied with figures are described in detail below.

### BRIEF DESCRIPTION OF THE DRAWINGS

FIG. 1 is a schematic side view of a sensing fiber according to the first embodiment of the invention.

FIG. 2A is a schematic cross-sectional view of the sensing fiber according to the first embodiment of the invention.

FIG. 2B is a partially enlarged view of the metal sensing layer in FIG. 2A.

FIG. 3A is a schematic cross-sectional view of a sensing fiber according to one embodiment of the invention.

FIG. 3B is a schematic cross-sectional view of a sensing fiber according to another embodiment of the invention.

FIG. 4A is a schematic view of a sensing device according to the second embodiment of the invention.

FIG. 4B is a partially enlarged view of the metal sensing layer in FIG. 4A.

FIG. 5A is a diagram about equivalent refractive index of surface plasmon mode and wavelength variation with different grating period of the third embodiment of the invention.

FIG. 5B is a graph about an imaginary part of equivalent refractive index of surface plasmon mode and wavelength variation in basic mode Ey direction of the third embodiment of the invention.

FIG. 6A is a diagram about equivalent refractive index of surface plasmon mode and wavelength variation with different metal materials of the forth embodiment of the invention.

FIG. 6B is the second graph about an imaginary part of equivalent refractive index of surface plasmon mode and wavelength variation with different metal materials in fundamental mode Ey direction of the fourth embodiment of the invention.

### DESCRIPTION OF THE EMBODIMENTS

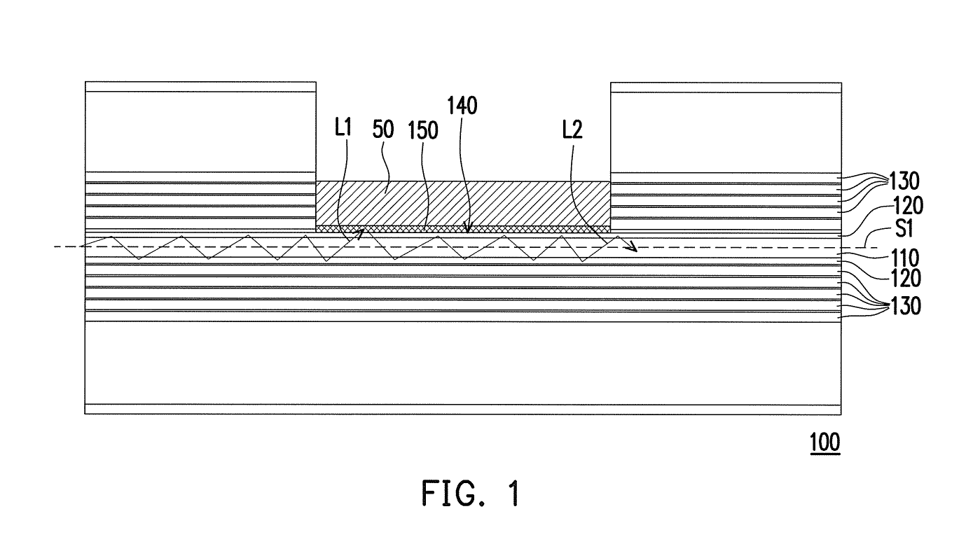

FIG. 1 is a schematic side view of a sensing fiber according to the first embodiment of the invention. In the first embodiment of the invention, a sensing fiber 100 is adapted to transmit a sensing light L1 along a path S 1 and senses an object 50. The sensing fiber 100 includes a core 110, a plurality of photonic crystal structures 120 and 130 surrounding the core 110, a sensing surface 140 and a metal sensing layer 150. The core 110 is located at the center of the sensing fiber 100. The photonic crystal structures 120 and 130 extend along the path S1.

Specifically, the sensing fiber 100 is, for example, a structure formed by machining a complete solid photonic crystal fiber, in fabricating process of the complete solid photonic crystal fiber, the solid columns, which are filled up with solid materials, are used to form the photonic crystal structures 120 and 130 of the complete solid photonic crystal fiber. In the present embodiment, the total number of coils of the photonic crystal structures 120 and 130 of the complete solid photonic crystal fiber is 5, but the total number of coils of the photonic crystal structure of the invention is not limited thereto.

In the first embodiment, the refractive coefficient of the photonic crystal structures 120 and 130 is smaller than the refractive coefficient of the core 110. Specifically, the refractive coefficient of the photonic crystal structures 120 and 130 ranges from 1.402 to 1.42, and the material of the core 110 is, for example, silica germanium which has the refractive coefficient ranging from 1.437 to 1.44, so that the refractive coefficient of the core is increased to make the sensing light L1 can be easily reflected inside the core 110 by the photonic crystal structures 120 and 130, and to increase the transmission efficiency.

In the present embodiment, the sensing surface 140 extends along a part of the path S 1 and be adjacent to the core 110. The sensing surface 140 in the present embodiment is, for example, formed by grinding and polishing the complete solid photonic crystal fiber. The metal sensing layer 150 is, for example, a film made by coating metal materials on the sensing surface 140. When the sensing fiber 100 senses the object 50, the metal sensing layer 150 is located between the sensing surface 140 and the object 50, and a part of the sensing light L1 is converted into a signal light L2 by the object 50 on the metal sensor layer 150.

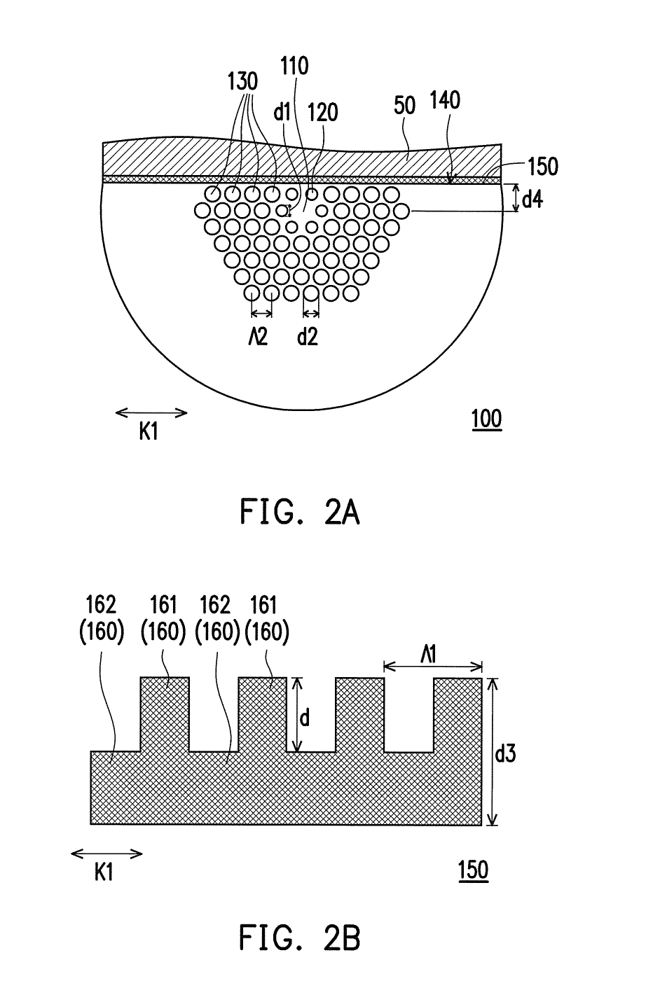

FIG. 2A is a schematic cross-sectional view of the sensing fiber according to the first embodiment of the invention. FIG. 2B is a partially enlarged view of the metal sensing layer in FIG. 2A. To be more specific, referring to FIG. 2A and FIG. 2B, the metal sensing layer 150 having a plurality of metal grating structures 160 is disposed on the sensing surface 140 in the present embodiment. Furthermore, the metal sensing layer 150 having the plurality of metal grating structures 160 has a distribution of thinner thickness and thicker thickness, so as to enhance the surface plasmon mode which is close to the sensing surface 140 when the sensing light L1 is transmitted inside the core 110, so that the object 50 is sensed more effectively by the sensing light L1. In other words, the distribution area of the surface plasmon mode on the metal sensing layer 150 can be increased by the metal grating structures 160, so that the sensing fiber 100 has a high transmission, a high sensitivity, and a low loss effect. On the other hand, the surface of the sensing fiber 100 is well coated by the metal sensing layer 150 because the sensing fiber 100 is formed by the complete photonic crystal fiber, so that the shape of the sensing fiber is not changed because of covering the air holes.

In the present embodiment, because the sensing surface 140 and metal sensing layer 150 of the sensing fiber 100 have a good surface plasmon mode, the sensing light L1 is sufficiently converted into a signal light L2 by the object 50 on the metal sensor layer 150. Specifically, in the present embodiment, a claw layer (not shown) is disposed on the metal sensing layer 150 and adjusted to combine with the object 50, the metal sensing layer 150 is located between the claw layer and the sensing surface 140. The claw layer is, for example, an antigen, and the object is, for example, an antibody. The signal light that is generated by conversion of the sensing light L1 received by the antibody individually and the signal light that is generated by conversion of the sensing light L1 received by the combination of the antibody and the antigen have different spectral distributions. Because of the different spectra, the sensing fiber 100 of the present embodiment is based on the signal light L2 to detect the existence of the antigen in the object 50, so as to provide a good sensing effect. Furthermore, the sensing fiber 100 of the present embodiment can be applied to the biosensor, and can sense the photoluminescence spectrum or the Raman spectrum of the object through the enhanced surface plasmon mode.

In the present embodiment, the metal sensing layer 150 further has a first metal layer 161 and a second metal layer 162 located between the sensing surface 140 and the first metal layer 161. Specifically, the metal sensing layer 150 is, for example, made by coating the sensing surface 140 with the first metal layer 161, the second metal layer 162 is then coated with the first metal layer 161, and the metal grating structure 160 is formed by etching the first metal layer 161 periodically so that the metal grating structures 160 are formed at the first metal layer 161.

Referring to FIG. 2A and FIG. 2B, in this embodiment, the metal grating structures 160 of the metal sensing layer 150 are arranged along a direction K1 perpendicular to the path S1. The metal grating structures 160 conform with 0.02≦d/Λ1≦0.04, where d is a depth of the metal grating structures 160 along a direction perpendicular to the sensing surface 140, and Λ1 is a pitch of the metal grating structures 160. On the other hand, the metal sensing layer 150 has a total thickness d3 in a direction perpendicular to the sensing surface 140, and the total thickness d3 is greater than or equal to 40 nm and less than or equal to 80 nm.

Specifically, the first metal layer 161 and the second metal layer 162 of the present embodiment all are silver films having a thickness of 40 nm, so as to fabricate the metal grating having periodic variation of height by etching the second metal layer 162 periodically, but the invention is not limited thereto. In other embodiments of the invention, the material of the metal sensing layer can further includes gold, copper, and silver.

Referring to FIG. 2A, in the first embodiment of the invention, a diameter dl of the photonic crystal structure 120 is equal to 1.2 micrometer (μm), and a diameter d 2 of the photonic crystal structure 130 is equal to 1.6 μm. To be more specific, the photonic crystal structure 120 can form an internal photonic crystal layer, the photonic crystal structure 130 can form an external photonic crystal layer, and the internal photonic crystal layer is located between the core 110 and the external photonic crystal layer. The diameter d1 of the cross-section of the photonic crystal structure 120 perpendicular to the path S1 (the cross-section is also depicted in FIG. 2A) and forming the internal photonic crystal layer is smaller than the diameter d2 of the cross-section of the photonic crystal structure 130 perpendicular to of the path S1 and forming the external photonic crystal layer. Furthermore, in an embodiment, the diameter of the cross-section of the photonic crystal structure perpendicular to the path and forming the internal photonic crystal layer is greater than or equal to 1.0 μm and less than or equal to 1.4 μm, and the diameter of the cross-section of the photonic crystal structure perpendicular to the path and forming the external photonic crystal layer is greater than or equal to 1.4 μm and less than or equal to 1.8 μm. In the present embodiment, the pitch Λ2 of the photonic crystal structure 130 is equal to 2 μm, but the invention is not limited thereto. In other embodiments of the invention, the pitch of the photonic crystal structures 120, 130 ranges from 2 to 2.6 μm.

Based on the above, the sensing fiber 100 of the present embodiment is foiined by grinding and polishing the complete solid photonic crystal fiber, a distance d 4 from the center of the core 110 to the sensing surface 140 is equal to 2.66 μm, but the invention is not limited thereto. In other embodiments of the invention, the distance between the sensing surface and the core ranges from 2 to 2.8 μm. On the other hand, in the present embodiment, the photonic crystal structures 120, 130 of the complete solid photonic crystal fiber are, for example, distributed to form a hexagonal distribution area inside the complete solid photonic crystal fiber, but the invention is not limited thereto.

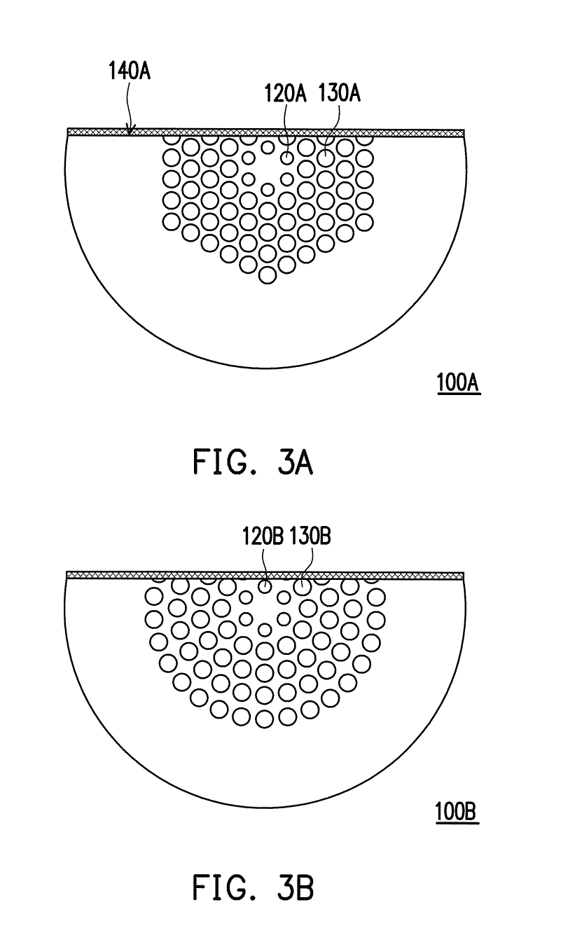

FIG. 3A is a schematic cross-sectional view of a sensing fiber according to one embodiment of the invention. Referring to FIG. 3A, in one embodiment of the invention, the sensing surface 140A of the sensing fiber 100A is formed by grinding and polishing from different directions towards the core. More specifically, the photonic crystal structures 120A, 130A of the present embodiment is a rotation by 90 degree about the center of the core of the photonic crystal structures 120, 130 of the first embodiment.

FIG. 3B is a schematic cross-sectional view of a sensing fiber according to another embodiment of the invention. Referring to FIG. 3B, in another embodiment of the invention, the photonic crystal structures 120B, 130B formed in the complete solid photonic crystal fiber of the sensing fiber 100B can further have a circular distribution in the fiber. Moreover, the cross section of the photonic crystal structures of the embodiments of the invention on a surface perpendicular to the path transmitting the sensing light of the sensing fiber can be arranged to foil u a semi-circular shape, a meniscus shape, or a polygonal shape.

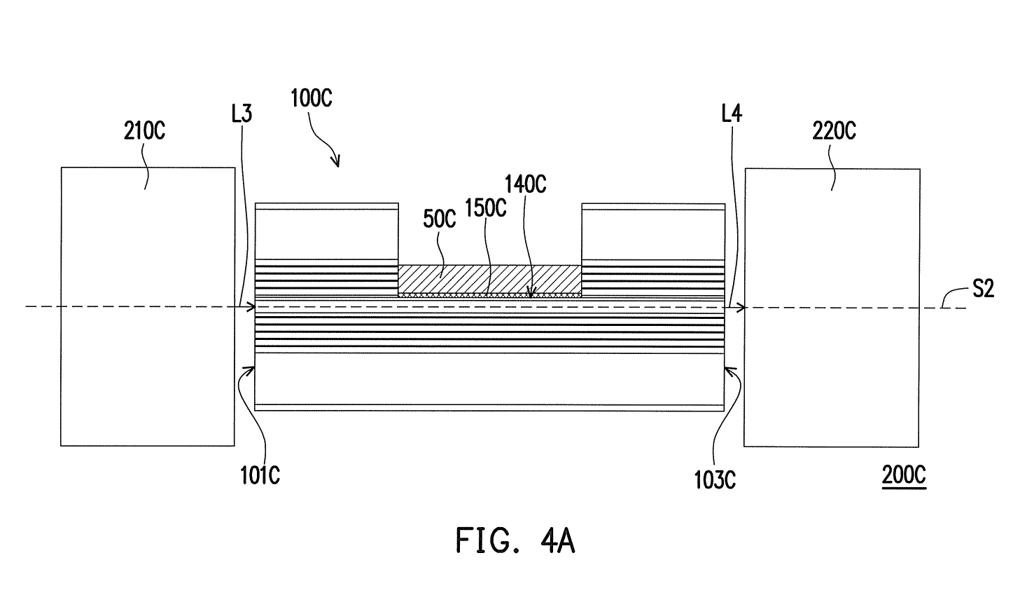

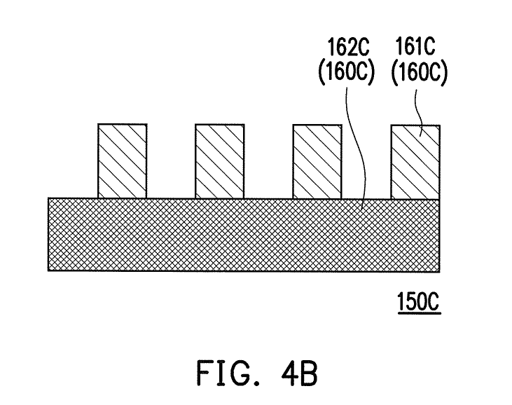

FIG. 4A is a schematic view of a sensing device according to the second embodiment of the invention. Referring to FIG. 4A, in the second embodiment of the invention, a sensing device 200C is adapted to sense an object 50c, and the sensing device 200C includes a light source 210C, the sensing fiber 100C, and a receiving unit 220C. The sensing fiber 100C is adapted to transmit the sensing light L3 emitted from the light source 210C along a path S2 and senses the object 50C. The sensing fiber 100C further includes a light-entering end 101C and a light-exiting end 103C, and the sensing surface 140C is located between the light-entering end 101C and the light-exiting end 103C. The sensing light L3 emitted by the light source 210C enters the sensing fiber 100C from the light-entering end 101C, a part of the sensing light L3 is converted into a signal light L4 by the object 50C on the metal sensor layer 150C, and the signal light L4 is emitted from the light-exiting end 103C and enters the receiving unit 220C.

In the present embodiment, the receiving unit is an optical spectrum analyzer, and the elements of the object can be manifested by the optical spectrum analyzer analyzing the spectrum of the signal light L4, but the invention is not limited thereto. In other embodiments of the invention, the receiving unit can be a power meter or a light meter.

FIG. 4B is a partially enlarged view of the metal sensing layer in FIG. 4A. Referring to FIG. 4B, the metal sensing layer 150C of the sensing fiber 100c in the present embodiment further includes a first metal layer 161C and a second metal layer 162C, and the material of the first metal layer 161C is different from the material of the second metal layer 162C. To be more specific, in the present embodiment, the material of the first metal layer 161C is silver, the material of the second metal layer 162C is copper, so as to form the metal sensing layer 150C having a plurality of metal grating structures 160C constructed by different metal materials, and simultaneously to increase the sensitivity of the sensing fiber.

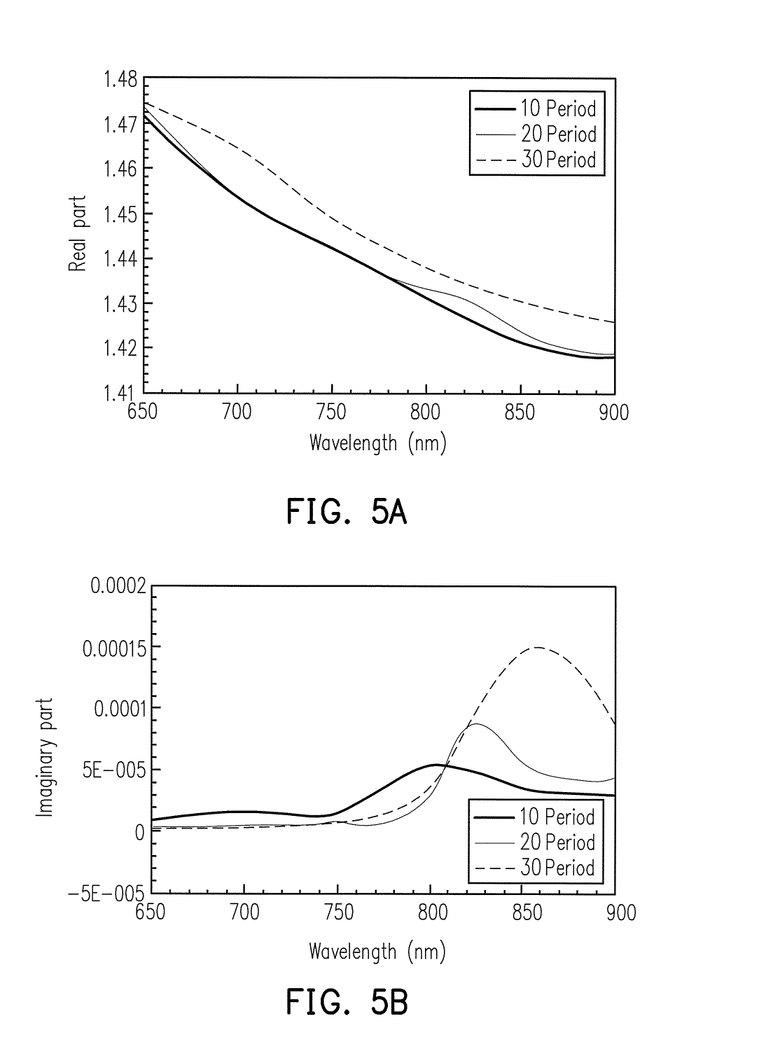

Table 1 contains the experimental data of the third embodiment of the invention, and FIG. 5A, 5B are graphs according to the experimental data of the metal sensing layer with each period of table 1. The mode real part and the mode imaginary part are calculated by the Lorentz model, and the sensitivity is calculated by the formula

$${S}_{λ} ( \frac{nm}{RIU} ) = \frac{d {λ}_{peak} ( {n}_{a} )}{d {n}_{a}} ,$$

wherein S_{λ} is the sensitivity having unit: nm/RIU (RIU is Refractive Index Unit), λₚₑₐₖ is the resonance wavelength when the coupled mode is generated, nₐ is the reflective index of the analyzed object.

TABLE 1
data in the coupled mode of 3 types of metal grating period

| Grating period | 10 | 20 | 30 |
|---|---|---|---|
| Period length (μm) | 3.7933 | 1.8966 | 1.2644 |
| Resonance wavelength (nm) | 800 | 820 | 850 |
| Basic mode real part | 1.431944 | 1.431488 | 1.430982 |
| Basic mode imaginary | 5.469743 | 8.606705 | 14.77885 |
| part (×10⁻⁵) | | | |
| Surface plasmon mode | 1.431355 | 1.431193 | 1.430977 |
| real part | | | |
| Surface plasmon mode | 0.004247 | 0.004516 | 0.003322 |
| imaginary part | | | |
| Sensitivity (μm/RIU) | 26.58 | 32.08 | 37.83 |

As shown in table 1, when the value of the period increases, the sensitivity also increases accordingly, so that the sensing fiber of the embodiments of the invention has the metal crystal structure which is arranged periodically, and the sensing fiber has a good sensitivity. FIG. 5A is a diagram about equivalent refractive index of surface plasmon mode and wavelength variation with different grating period of the third embodiment of the invention. It is observed in FIG. 5A that the sensing fiber having the metal grating structure has a surface plasmon mode with a good equivalent refractive index. FIG. 5B is a graph about an imaginary part of equivalent refractive index of surface plasmon mode and wavelength variation in basic mode Ey direction of the third embodiment of the invention. It is observed in FIG. 5B that the metal grating structures which are arranged periodically can enhance the equivalent refractive index of the surface plasmon mode, the loss is increased accordingly, and the field distribution in the coupled mode is relatively clearer.

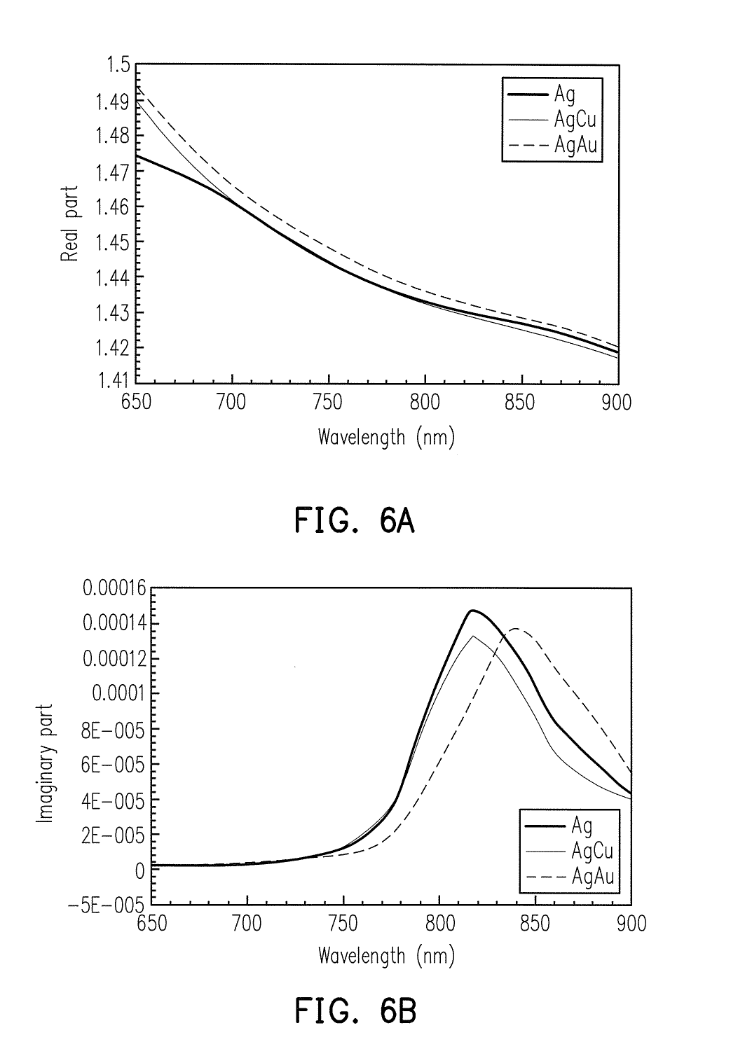

Table 2 contains the experimental data of the fourth embodiment of the invention, and FIG. 6A, 6B are graphs according to the experimental data of the metal sensing layer with each period of table 2. The mode real part and the mode imaginary part are calculated by the Lorentz model, and the sensitivity is calculated by the formula

$${S}_{λ} ( \frac{nm}{RIU} ) = \frac{d {λ}_{peak} ( {n}_{a} )}{d {n}_{a}} ,$$

wherein S_{λ} is the sensitivity having unit: nm/RIU (RIU is Refractive Index Unit), λₚₑₐₖ is the resonance wavelength when the coupled mode is generated, nₐ is the reflective index of the analyzed object.

TABLE 2
data in the coupled mode of 3 types of metal grating

| Grating metal materials | Ag | Ag—Cu | Ag—Au |
|---|---|---|---|
| Period length (μm) | 1.2644 | | |
| Resonance wavelength (nm) | 850 | 850 | 875 |
| Basic modal real part | 1.43092 | 1.43101 | 1.430443 |
| Basic modal imaginary part | 14.77885 | 13.34496 | 13.69802 |
| (×10⁻⁵) | | | |
| Surface plasmon mode | 1.430977 | 1.430074 | 1.430214 |
| real part | | | |
| Surface plasmon mode | 0.003322 | 0.00356 | 0.003705 |
| imaginary part | | | |
| Sensitivity (μm/RIU) | 37.83 | 37.83 | 40.71 |

In detail, FIG. 6A is a diagram about equivalent refractive index of surface plasmon mode and wavelength variation with different metal materials of the forth embodiment of the invention, FIG. 6B is the second graph about an imaginary part of equivalent refractive index of surface plasmon mode and wavelength variation with different metal materials in fundamental mode Ey direction of the fourth embodiment of the invention. It is observed in FIG. 6A that the equivalent refractive index of the surface plasmon mode of the metal sensing layer is higher than the other two materials, and it is discovered in FIG. 6B that the metal sensing layer has a greater loss and simultaneously has a high sensitivity.

In summary, the sensing fiber of the embodiments of the invention has different metal sensing layers disposed on the sensing surface, the metal sensing layer has the plurality of the metal grating structures. When the sensing light is transmitted in the sensing fiber, the sensing fiber can have a good surface plasmon mode, so that the sensing light can sense the object on the metal sensing layer effectively, the signal light converted by the analyzed object is obtained to provide a good sensing effect. In other words, the sensing fiber of the embodiments of the invention are combined with the evanescent wave of the fiber and the metallic grating structure for the two kinds of generating mechanism of surface plasmon mode, so as to increase the sensitivity and practicality of the sensing fiber. Because the sensing device of the embodiments of the invention has the sensing fiber, when the light source emits the sensing light to the sensing fiber, the receiving unit can all receive a good signal light of the object.

It will be apparent to those skilled in the art that various modifications and variations can be made to the structure of the disclosed embodiments without departing from the scope or spirit of the invention. In view of the foregoing, it is intended that the invention cover modifications and variations of this invention provided they fall within the scope of the following claims and their equivalents.

What is claimed is:

1. A sensing fiber adapted to transmit a sensing light along a path and sensing an object, the sensing fiber comprising:
a core, located at a center of the sensing fiber;
a plurality of photonic crystal structures, surrounding the core and extending along the path;
a sensing surface, extending along part of the path and being adjacent to the core; and
a metal sensing layer, disposed on the sensing surface, wherein the metal sensing layer has a plurality of metal grating structures, when the sensing fiber senses the object, the metal sensing layer is located between the sensing surface and the object, and a part of the sensing light is converted into a signal light by the object on the metal sensor layer.

2. The sensing fiber as recited in claim 1, wherein the metal grating structures of the metal sensing layer are arranged along a direction perpendicular to the path.

3. The sensing fiber as recited in claim 1, wherein the metal sensing layer has a total thickness in a direction perpendicular to the sensing surface, the total thickness is greater than or equal to 40 nm and less than or equal to 80 nm.

4. The sensing fiber as recited in claim 1, wherein the metal sensing layer further has a first metal layer and a second metal layer, the second metal layer is located between the sensing surface and the first metal layer, the metal grating structures are formed at the first metal layer.

5. The sensing fiber as recited in claim 1, wherein the metal grating structures conform with
$$0.02 \leq \frac{d}{Λ} \leq 0.04 ,$$
wherein d is a depth along a direction perpendicular to the sensing surface o f the metal grating structures, Λ is a pitch of the metal grating structures.

6. A sensing device adapted to sense an object, the sensing device comprising:
a light source, adapted to emit a sensing light;
a sensing fiber, adapted to transmit the sensing light along a path and sensing an object, the sensing fiber comprising:
a light-entering end;
a light-exiting end;
a core, located at a center of the sensing fiber;
a plurality of photonic crystal structures, surrounding the core and extending along the path;
a sensing surface, extending along a part of the path and being adjacent to the core, wherein the sensing surface is located between the light-entering end and the light-exiting end; and
a metal sensing layer, disposed on the sensing surface, wherein the metal sensing layer has a plurality of metal grating structures, and wherein when the sensing fiber senses the object, the metal sensing layer is located between the sensing surface and the object; and
a receiving unit, wherein the sensing light emitted by the light source enters the sensing fiber from the light-entering end, a part of the sensing light is converted into a signal light by the object on the metal sensor layer, and the signal light is emitted from the light-exiting end and enters the receiving unit.

7. The sensing device as recited in claim 6, wherein the metal sensing layer has a total thickness in a direction perpendicular to the sensing surface, the total thickness is greater than or equal to 40 nm and less than or equal to 80 nm.

8. The sensing device as recited in claim 6, wherein the metal sensing layer further comprises a first metal layer and a second metal layer, the second metal layer is located between the sensing surface and the first metal layer, and the metal grating structures are formed at the first metal layer.

9. The sensing device as recited in claim 6, wherein the metal grating structures conform with
$$0.02 \leq \frac{d}{Λ} \leq 0.04 ,$$
wherein d is a depth o f the metal grating structures along a direction perpendicular to the sensing surface, Λ is a pitch of the metal grating structures.

10. The sensing device as recited in claim 6, wherein the receiving unit is an optical spectrum analyzer, a power meter, or a light meter.
